(19) United States
(12) Patent Application Publication
(10) Pub. No.: US 2007/0238674 A1
(43) Pub. Date: Oct. 11, 2007

(54) TRA COMBINATION THERAPIES

(76) Inventors: Enrico P. Veltri, Princeton, NJ (US); William J. Greenlee, Teaneck, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(21) Appl. No.: 11/696,898

(22) Filed: Apr. 5, 2007

Related U.S. Application Data
(60) Provisional application No. 60/790,469, filed on Apr. 6, 2006.
(60) Provisional application No. 60/808,611, filed on May 26, 2006.
(60) Provisional application No. 60/809,785, filed on May 31, 2006.
(60) Provisional application No. 60/839,474, filed on Aug. 23, 2006.
(60) Provisional application No. 60/839,484, filed on Aug. 23, 2006.
(60) Provisional application No. 60/887,236, filed on Jan. 30, 2007.

(51) Int. Cl. A61K 31/704; A61K 31/554; A61K 31/4433; A61K 31/455; A61K 31/401; A61K 31/366; A61K 31/22; A61K 31/138

(52) U.S. Cl. 514/26; 514/337; 514/355; 514/252.17; 514/305; 514/457; 514/262.1; 514/651; 514/423; 514/460; 514/548; 514/211.07

(57) ABSTRACT

Disclosed herein are pharmaceutical combinations comprising at least one thrombin receptor antagonist and at least one cardiovascular agent. Examples of such a thrombin receptor antagonist include: 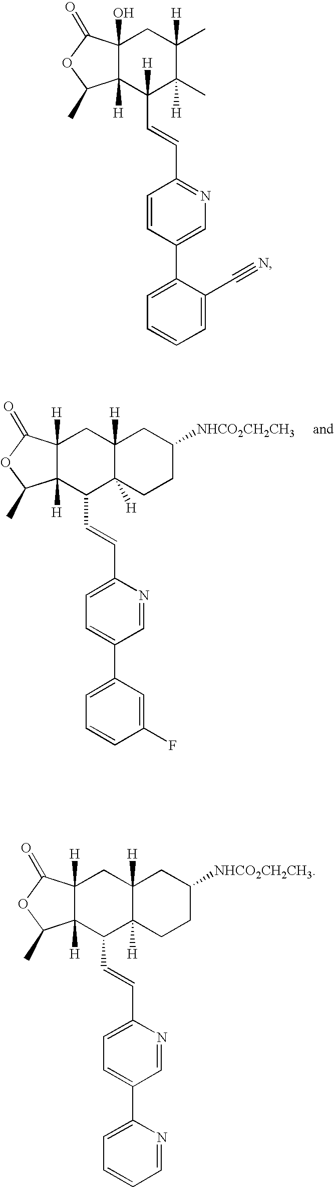 Examples of cardiovascular agents suitable for co-formulation or co-administration with the thrombin receptor antagonist include an endothelin antagonist selected from the group consisting of tezosentan, bosentan, and sitaxsentan.

### CROSS-REFERENCE TO RELATED TO APPLICATIONS

This application claims the benefit of the following U.S. provisional application Nos. 60/790,469, filed on Apr. 6, 2006; 60/808,611, filed on May 26, 2006; 60/809,785, filed on May 31, 2006; 60/839,474, filed on Aug. 23, 2006; 60/839,484, filed on Aug. 23, 2006; and, 60/887,236, filed on Jan. 31, 2007, all of which are herein incorporated by reference.

### BACKGROUND OF THE INVENTION

Thrombin is known to have a variety of activities in different cell types and thrombin receptors are known to be present in such cell types as human platelets, vascular smooth muscle cells, endothelial cells and fibroblasts.

Thrombin receptor antagonists (“TRAs”) have been identified based on structure-activity studies involving substitutions of amino acids on thrombin receptors. In Bernatowicz et al, J. Med. Chem., vol. 39, pp. 4879-4887 (1996), tetra- and pentapeptides are disclosed as being potent thrombin receptor antagonists, for example N-trans-cinnamoyl-p-fluoroPhe-p-guanidinoPhe-Leu-Arg-NH₂ and N-trans-cinnamoyl-p-fluoroPhe-p-guanidinoPhe-Leu-Arg-Arg-NH₂. Peptide thrombin receptor antagonists are also disclosed in WO 94/03479, published Feb. 17, 1994.

Thrombin receptor antagonists have been suggested in the literature as being potentially useful in treating a variety of cardiovascular diseases or conditions including, for example, thrombosis, vascular restenosis, deep venous thrombosis, lung embolism, cerebral infarction, heart disease, disseminated intravascular coagulation syndrome, hypertension (Suzuki, Shuichi, PCT Int. Appls. WO 0288092 (2002), WO 0285850 (2002) and WO 0285855 (2002)), arrhythmia, inflammation, angina, stroke, atherosclerosis, ischemic conditions (Zhang, Han-cheng, PCT Int. Appl. WO 0100659 (2001), WO 0100657 (2001) and WO 0100656 (2001)).

Substituted thrombin receptor antagonists are disclosed in U.S. Pat. Nos. 6,063,847; 6,326,380; and 6,645,987 and U.S. publication nos. 03/0203927; 04/0216437A1; 04/0152736; and 03/0216437. The use of a small subset of thrombin receptor antagonists to treat a variety of conditions and diseases is disclosed in U.S. publication no, 04/0192753. The prevention of complications associated with cardiopulmonary bypass surgery by administration of a thrombin receptor antagonist is taught in U.S. application Ser. No. 11/613,450. A crystalline form of the bisulfate salt of a particular thrombin receptor antagonist (identified as “compound A” below) is disclosed in 04/0176418A1.

In the search for enhanced efficacy and safety, researchers have explored various therapeutic combinations of two or more distinct active pharmaceutical agents. Such agents may act by very different biochemical pathways to provide particularly beneficial therapeutic results. The two or more active agents may be delivered as either co-administered individual formulations, or as a single co-formulation. Co-formulations have the patient-compliance advantages of reducing the number of distinct doses and fixing the ratio of the two active agents being administered. An example of such a combination is Vytorin®, which is a single dosage form comprising simvastatin (marketed in the U.S. as a monotherapy as Zocor®) and ezetimibe (marketed in the U.S. as a monotherapy as Zetia®).

### SUMMARY OF THE INVENTION

The present invention is directed to pharmaceutical compositions comprising an effective amount of at least one thrombin receptor antagonist, an effective amount of at least one cardiovascular agent selected from the group consisting of calcium channel blockers, statins, cholesterol absorption inhibitors, low molecular weight heparins, antiarrhythmic agents, alpha adrenergic agonists, beta adrenergic blocking agents, aidosterone antagonists, angiotensin-converting-enzyme (“ACE”) inhibitors, ACEINEP inhibitors, angiotensin II receptor blockers (“ARBs”), endothelin antagonists, neutral endopeptidase inhibitors, phosphodiesterase inhibitors, fibrinolytics, GP IIb/IIIa antagonists, direct thrombin inhibitors, indirect thrombin inhibitors, lipoprotein-associated phospholipase A2 (“LpPLA₂”) modulators, direct factor Xₐ inhibitors, indirect factor Xₐ inhibitors, indirect factor Xₐ/IIₐ inhibitors, diuretics, nitrates, thromboxane antagonists, platelet aggregations inhibitors, cyclooxygenase inhibitors, B-type natriuretic peptides, NV1FGF modulators, HT1B/5-HT2A antagonists, guanylate cyclase activators, e-NOS transcription enhancers, anti-atherogenics, CPU inhibiters, renin inhibitors, inhibitors of adenosine diphosphate (“ADP”)-induced platelet aggregation, and NHE-1 inhibitors, and

a pharmaceutically acceptable carrier for the treatment of a condition in a mammal.
In some embodiments, the thrombin receptor antagonist is selected from the group consisting of 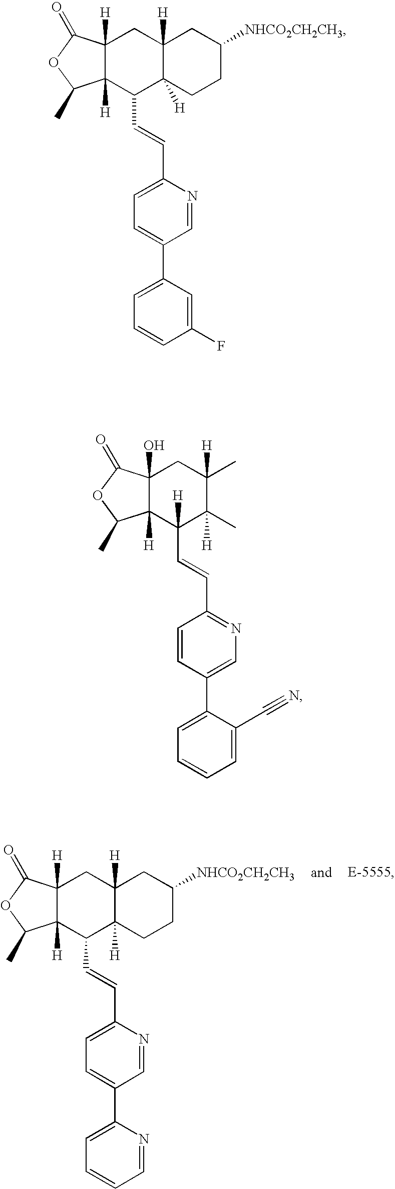
or a pharmaceutically acceptable isomer, salt, solvate or co-crystal thereof.

In some embodiments, the thrombin receptor antagonist is 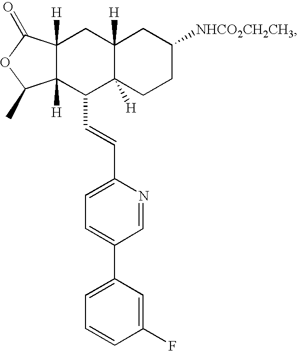
or a pharmaceutically acceptable isomer, salt, solvate or co-crystal thereof. In some embodiments, the salt is a bisulfate salt.
In some embodiments, the thrombin receptor antagonist is 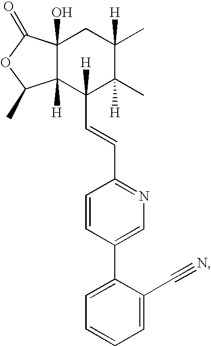
or a pharmaceutically acceptable isomer, salt, solvate or co-crystal thereof.

In some embodiments, the thrombin receptor antagonist is 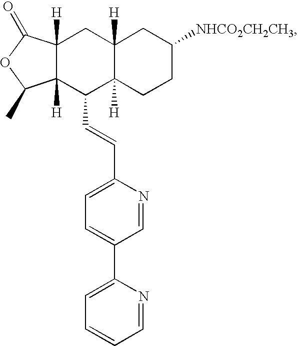
or a pharmaceutically acceptable isomer, salt, solvate or co-crystal thereof.

In some embodiments, the thrombin receptor antagonist is E-5555.

In some embodiments, the cardiovascular agent is a calcium channel blocker selected from the group consisting of amlodipine, felodipine, diltiazem, verapamil, nifedipine, nicardipine, nisoldipine, bepridil, and verapamil.

In some embodiments, the cardiovascular agent is a statin selected from the group consisting of atorvastatin, fluvastatin, lovastatin, pitavastatin, pravastatin, rosuvastatin, and simvastatin.

In some embodiments, the cardiovascular agent is a cholesterol absorption inhibitor selected from the group consisting of ezetimibe and AZD 4121.

In some embodiments, the cardiovascular agent is a low molecular weight heparin selected from the group consisting of dalteparin, ardeparin, certoparin, enoxaparin, parnaparin, tinzaparin, reviparin, nadroparin, warfarin, ximelagatran, fondaparin, and enoxaparin.

In some embodiments, the cardiovascular agent is an antiarrhythmic agent selected from the group consisting of dofetilide, ibutilide, metoprolol, propranolol, atenolol, ajmaline, disopyramide, prajmaline, procainamide, quinidine, sparteine, aprindine, lidocaine, mexiletine, tocamide, encamide, flecamide, lorcamide, moricizine, propafenone, acebutolol, pindolol, amiodarone, bretylium, bunaftine, dofetilide, sotalol, adenosine, atropine and digoxin.

In some embodiments, the cardiovascular agent is an alpha adrenergic agonist selected from the group consisting of doxazosin, terazoson and prazosin.

In some embodiments, the cardiovascular agent is a beta adrenergic blocking agent selected from the group consisting of carvedilol, propranolol, timolol, nadolol, atenolol, metoprolol, bisoprolol, nebivolol, betaxolol, acebutolol, and bisoprolol.

In some embodiments, the cardiovascular agent is an ACE inhibitor selected from the group consisting of moexipril, quinapril ramipril, lisinopril, benazapril, enalapril, captopril, spirapril, perindopril, fosinopril and trandolapril.

In some embodiments, the cardiovascular agent is an ARB selected from the group consisting of olmesartan, candesartan, valsartan, telmisartan, irbesartan, losartan and eprosartan.

In some embodiments, the cardiovascular agent is an endothelin antagonist selected from the group consisting of tezosentan, bosentan, and sitaxsentan.

In some embodiments, the cardiovascular agent is a direct thrombin inhibitor selected from the group consisting of ximelagatran and AZDO837.

In some embodiments, the cardiovascular agent is a direct factor Xₐ inhibitor selected from the group consisting of fondaparinux, apixaban, razaxaban, rivaroxaban, KFA-1982, DX-9065a, AVE3247, otamixaban, AVE6324 and SAR377142.

In some embodiments, the cardiovascular agent is an inhibitor of adenosine diphosphate (“ADP”)-induced platelet aggregation selected from the group consisting of clopidogrel, ticlopidine, prasugrel, and AZD6140.

In other embodiments, the invention comprises a method of treating or preventing a cardiovascular condition in a mammal in need thereof comprising administering to said mammal any of the above pharmaceutical compositions, wherein said cardiovascular condition is acute coronary syndrome, secondary prevention, peripheral arterial disease, thrombosis, atherosclerosis, restenosis, hypertension, angina pectoris, arrhythmia, heart failure, myocardial infarction, glomerulonephritis, thrombotic stroke, thromboembolic stroke, deep vein thrombosis, venous thromboembolism, a cardiovascular disease associated with hormone replacement therapy, disseminated intravascular coagulation syndrome, renal ischemia, cerebral stroke, cerebral ischemia, cerebral infarction, migraine, renal vascular homeostasis or erectile dysfunction.

In other embodiments, the invention comprises a method of treating or preventing a cardiovascular condition in a mammal in need of said treating comprising administering to said mammal a first pharmaceutical composition comprising a thrombin receptor antagonist and a second pharmaceutical composition comprising a cardiovascular agent.

In some of these embodiments, the thrombin receptor antagonist is 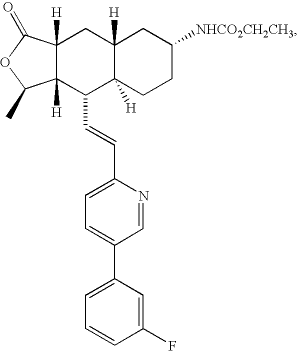
or a pharmaceutically acceptable isomer, salt, solvate or co-crystal thereof.

In some of these embodiments, the cardiovascular agent is selected from the group consisting of calcium channel blockers, statins, cholesterol absorption inhibitors, low molecular weight heparins, antiarrhythmic agents, alpha adrenergic agonists, beta adrenergic blocking agents, aldosterone antagonists, angiotensin-converting-enzyme (“ACE”) inhibitors, ACE/NEP inhibitors, angiotensin II receptor blockers (“ARBs”), endothelin antagonists, neutral endopeptidase inhibitors, phosphodiesterase inhibitors, fibrinolytics, GP IIb/IIIa antagonists, direct thrombin inhibitors, indirect thrombin inhibitors, lipoprotein-associated phospholipase A2 (“LpPLA₂”) modulators, direct factor Xₐ inhibitors, indirect factor Xₐ inhibitors, indirect factor Xₐ/IIₐ inhibitors, diuretics, nitrates, thromboxane antagonists, platelet aggregations inhibitors, cyclooxygenase inhibitors, B-type natriuretic peptides, NV1FGF modulators, HT1B/5-HT2A antagonists, guanylate cyclase activators, e-NOS transcription enhancers, anti-atherogenics, CPU inhibiters, renin inhibitors, inhibitors of adenosine diphosphate (“ADP”)-induced platelet aggregation, and NHE-1 inhibitors.

### DETAILED DESCRIPTION

As used throughout the specification, the following terms, unless otherwise indicated, shall be understood to have the following meanings:

“Subject” includes both mammals and non-mammalian animals.

“Mammal” includes humans and other mammalian animals.

“Effective amount” or “therapeutically effective amount” is meant to describe an amount of compound or a composition of the present invention effective in producing the desired therapeutic, ameliorative, inhibitory or preventative effect. For example, an effective amount of a thrombin receptor antagonist is an amount sufficient to achieve a desired threshold of PAR-1 antagonism.

“Acute coronary syndrome” includes any group of clinical symptoms compatible with acute myocardial ischemia. Acute myocardial ischemia is chest pain due to insufficient blood supply to the heart muscle that results from coronary artery disease (also called coronary heart disease). Acute coronary syndrome thus covers the spectrum of clinical conditions ranging from unstable angina to non-O-wave myocardial infarction and Q-wave myocardial infarction. Symptoms may include chest pain, shortness of breath, nausea, vomiting, diaphoresis (sweating), palpitations, anxiety or a sense of impending doom and a feeling of being acutely ill.

“Secondary prevention” means treating patients who have already suffered a significant cardiovascular event, such as a heart attack or stroke, to prevent another future, potentially more serious, perhaps lethal, cardiovascular or cerebrovascular event.

“MI” mean myocardial infarction.

“CABG” means coronary bypass graft.

“PCI” means percutaneous coronary intervention, commonly known as coronary angioplasty, is an invasive cardiologic therapeutic procedure to treat the stenotic (narrowed) coronary arteries of the heart. These stenotic segments are due to the build up of cholesterol-laden plaques that form due to coronary heart disease. PCI, as used herein, will be understood to include balloon angioplasty, implantation of stents, rotational or laser atherectomy, and brachytherapy.

A variety of compounds have been demonstrated as displaying activity as thrombin receptor antagonists, many being himbacine analogs. As disclosed in U.S. publication no. 04/0152736, a subset of particularly preferred compounds of Formula I is as follows: 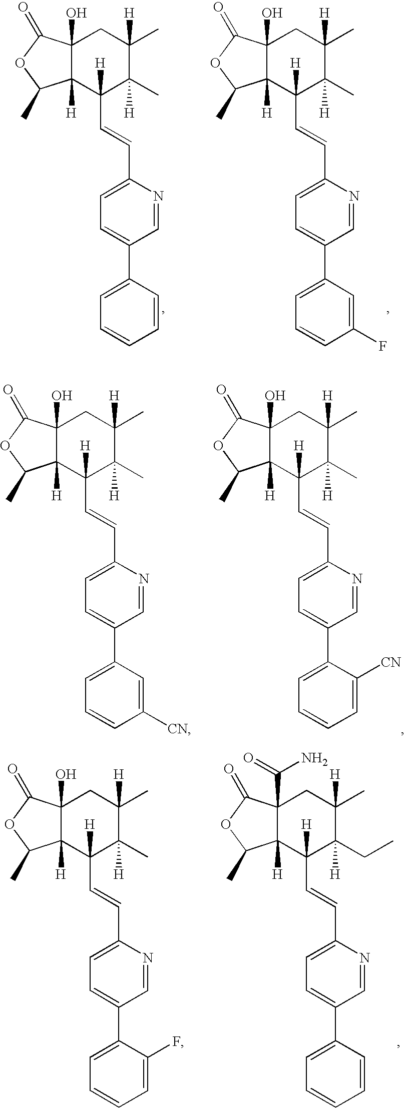 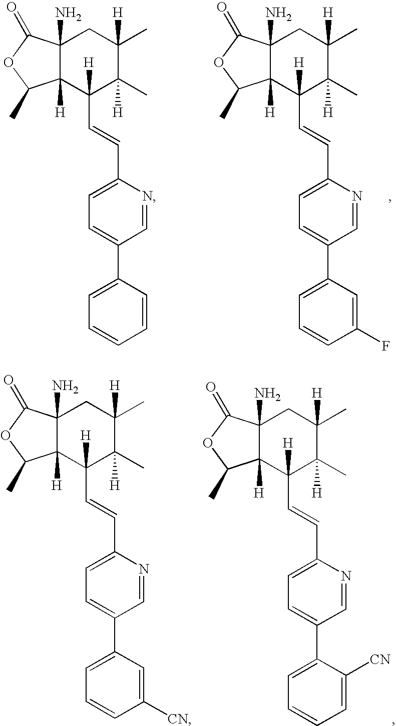
and the pharmaceutically acceptable isomers, salts, solvates and polymorphs thereof. U.S. publication no. 03/0216437 discloses a subset of thrombin receptor antagonists of Formula II which are both particularly active and selective. These compounds are as follows: 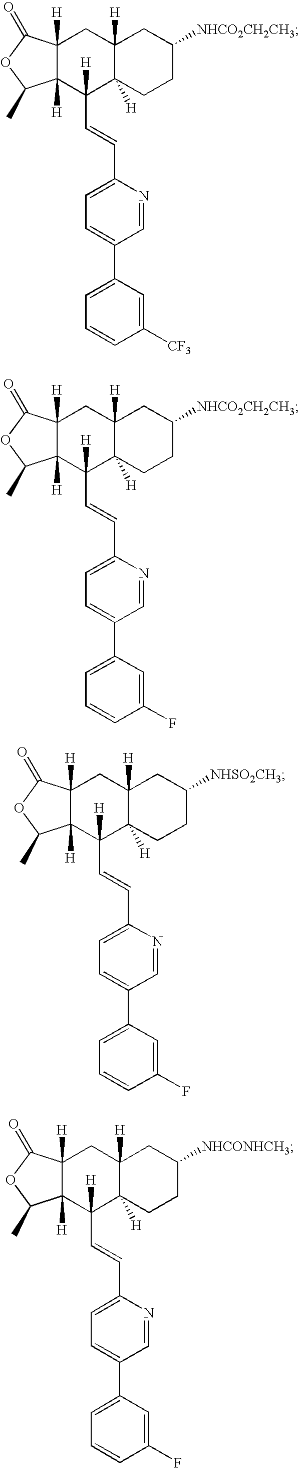 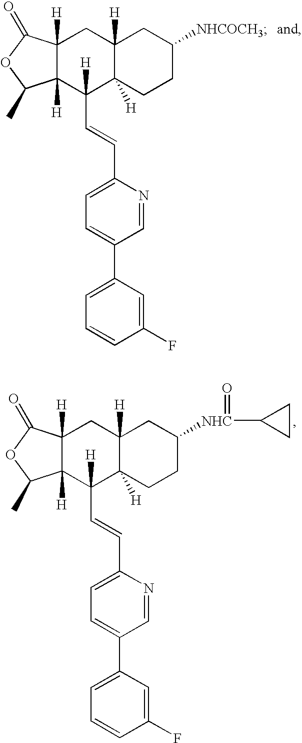
and the pharmaceutically acceptable isomers, salts, solvates and polymorphs thereof.

The following compounds are particularly favored based on their pharmacokinetics and phamacodynamic characteristics: 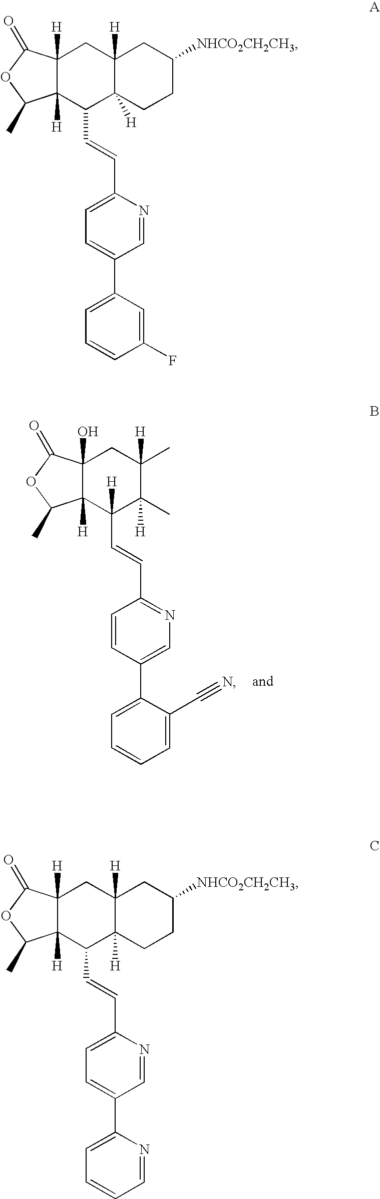
and the pharmaceutically acceptable isomers, salts, solvates and polymorphs thereof. The bisulfate salt of Compound A is currently in development as a thrombin receptor antagonist by Schering Corp. Its synthesis is disclosed in U.S. publication no. 03/0216437, published Nov. 20, 2003, which publication also discloses Compound C. Compound B is disclosed in U.S. Pat. No. 6,645,987.

Other compounds for use in the combinations of the present invention are disclosed in any of U.S. Pat. Nos. 6,063,847 and 6,326,380, U.S. Patent Publications 03/0203927, 03/0216437, 04/0192753 and 04/0176418, all of which are incorporated by reference in their entirety. Combinations that include one or more other agents that display activity as thrombin receptor antagonists are also within the scope of the present invention, including E5555 currently in development by Eisai:

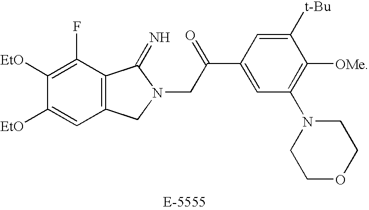

The compounds for use in the combinations of the present invention can form salts, the pharmaceutically acceptable of which are also within the scope of this invention.

Therapeutic Combinations

The therapeutic combinations of the present invention encompass at least one thrombin receptor antagonist along with at least one additional therapeutically effective agent. The thrombin receptor antagonist can be chosen from any of those disclosed herein, or from other compounds which display activity as thrombin receptor antagonists.

The therapeutically effective agents may be cardiovascular agents. In particular, cardiovascular agents that can be used in combination with the thrombin receptor antagonist include drugs that have anti-thrombotic, anti-platelet aggregation, antiatherosclerotic, antirestenotic and/or anti-coagulant activity. Such drugs are useful in treating thrombosis-related diseases including thrombosis, atherosclerosis, restenosis, hypertension, angina pectoris, arrhythmia, heart failure, myocardial infarction, glomerulonephritis, thrombotic and thromboembolic stroke, peripheral vascular diseases, other cardiovascular diseases, cerebral ischemia, inflammatory disorders and cancer, as well as other disorders in which thrombin and its receptor play a pathological role.
Suitable cardiovascular agents are selected from one or more of the group consisting of:

calcium channel blockers, such as amlodipine besilate, marketed as NORVASC® and LOTREL®, felodipine, marketed as PLENDIL®, diltiazem, marketed as CARDIZEM®, verapamil, marketed as CALAN®, nifedipine, marketed as ADALAT®, nicardipine, marketed as CARDENE®, nisoldipine, marketed as SULAR®, bepridil, marketed as VASCOR®, and verapamil, marketed as CALAN®;

Iomerizine, used as an anti-hypertensive and in the treatment of angina;

statins, such as atorvastatin, marketed as LIPITOR®, fluvastatin, marketed as LESCOL®, lovastatin, marketed as MEVACOR®, pitavastatin, marketed as LIVALO®, pravastatin, marketed as PRAVACHOL®, rosuvastatin, marketed as CRESTOR®, and simvastatin, marketed as ZOCOR®, used to treat high cholesterol (LDL);

cholesterol absorption inhibitors, such as ezetimibe, marketed as ZETIA® and AZD4121, which is in development for dyslipidaemia;

cholesteryl ester transfer protein (“CETP”) inhibitors, such as torcetrapib;

low molecular weight heparins, such as dalteparin sodium, marketed as FRAGMIN®, ardeparin, marketed as NORMIFLO®, certoparin, marketed as SANDOPARIN®, enoxaparin, marketed as LOVENOX® and CLEXANE®, parnaparin, marketed as FLUXUM®, tinzaparin, marketed as INNOHEP® and LOGIPARIN®, reviparin, marketed as CLIVARIN®, nadroparin, marketed as FRAXIPARIN®, warfarin, ximelagatran, fondaparin, and enoxaparin, used for prophylaxis or treatment of deep vein thrombosis and pulmonary embolism;

antiarrhythmic agents, such as dofetilide and ibutilide fumarate, marketed as TIKOSYN®, metoprolol, marketed as TOPROL-XL®, metoprolol tartrate, marketed as LOPRESSOR®, propranolol, marketed as INDERAL®, atenolol, marketed as TENORMIN®, ajmaline, disopyramide, prajmaline, procainamide, quinidine, sparteine, aprindine, lidocaine, mexiletine, tocamide, encamide, flecamide, lorcamide, moricizine, propafenone, acebutolol, pindolol, amiodarone, bretylium tosylate, bunaftine, dofetilide, sotalol, adenosine, atropine and digoxin, used for the maintenance of sinus rhythm in individuals prone to the formation of atrial fibrillation and flutter, and for the chemical cardioversion to sinus rhythm from atrial fibrillation and flutter;

alpha adrenergic agonists, such as doxazosin mesylate, marketed as CARDURA®, terazoson, marketed as HYTRIN®, and prazosin, marketed as MINIPRESS®, used to treat hypertension;

beta adrenergic blocking agents, such as carvedilol, marketed as COREG®, propranolol, marketed as BETACHRON E-R®, timolol, marketed as BLOCADREN®, nadolol, marketed as CORGARD®, atenolol, marketed as TENORMIN®, metoprolol, marketed as TOPROL XL®, bisoprolol, marketed as ZEBETA®, nebivolol, betaxolol, marketed as KERLONE®; acebutolol, marketed as SECTRAL, and bisoprolol, marketed as ZEBETA;

aldosterone antagonists, such as eplerenone, marketed as INSPRA®, and spironolactone, marketed as Aldactone®, used for reducing cardiovascular risk in patients following myocardial infarction;

angiotensin-converting-enzyme (“ACE”) inhibitors, such as moexipril, marketed as UNIVASC®, quinapril hydrochloride, marketed as ACCUPRIL®, ramipril, marketed as RAMACE® and ALTACE®, lisinopril, marketed as ZESTRIL®, benazepril hydrochloride, marketed as LOTENSIN®, enalapril, marketed as VASOTEC®, captopril, marketed as CAPOTEN®, spirapril, perindopril, marketed as ACEON®, fosinopril, marketed as MONOPRIL®, and trandolapril, marketed as MAVIK®, used to treat hypertension;

ACE/NEP inhibitors, such as ramipril, marketed as DELIX®JTRITACE®;

angiotensin II receptor blockers (“ARBs”), such as olmesartan medoxomil, marketed as BENICAR®, candesartan, marketed as ATACAND®, valsartan, marketed as DIOVAN®, telmisartan, marketed as MICARDIS®, irbesartan, marketed as AVAPRO®, losartan, marketed as COZAAR®, and eprosartan, marketed as TEVETEN®, used for treatment of hypertension;

endothelin antagonists, such as tezosentan, bosentan, marketed as TRACLEER®, and sitaxsentan sodium, to be marketed as THELIN®;

neutral endopeptidase inhibitors, such as candoxatril and ecadotril;

phosphodiesterase inhibitors, such as milrinoone, theophylline, vinpocetine, EHNA (erythro-9-(2-hydroxy-3-nonyl)adenine), sildenafil citrate, marketed as VIAGRA®, and tadalafil, marketed as CIALIS®;

fibrinolytics, such as reteplase, alteplase, and tenecteplase;

GP IIb/IIIa antagonists, such as integrillin, abciximab, and tirofiban;

direct thrombin inhibitors, such as ximelagatran, which has been marketed as EXANTA®, and AZD0837, which is in development for thrombosis:

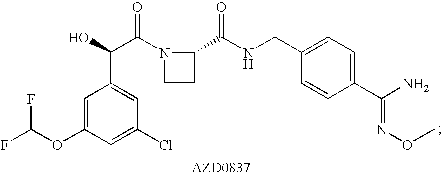

indirect thrombin inhibitors, such as odiparcil, currently in development by GlaxoSmithKline for prevention of thrombotic complications of cardiovascular disease;

lipoprotein-associated phospholipase A2 (“LpPLA₂”) modulators;

direct factor Xₐ inhibitors, such as fondaparinux sodium, marketed as ARIXTRA®, apixaban, razaxaban, INDUPRUX®, rivaroxaban (BAY 59-7939), KFA-1982, DX-9065a, AVE3247, otamixaban (XRP0673), AVE6324 and SAR377142;

indirect Xₐ inhibitors, such as idraparinux (long-acting pentasaccharide), fondaparinux sodium (pentasaccharide) and SSR126517;

indirect Xₐ/IIₐ inhibitors, such as enoxaparin sodium, marketed as SR123781 (short-acting hexadecasaccharide), AVE5026, SSR128428 (long-acting hexadecasaccharide), and SSR128429;

diuretics, such as chlorthalidone, a component in the combination marketed as TENORETIC®, ethacrynic acid, furosemide, amiloride, chlorothiazide, marketed as DIURlL®), hydrochlorothiazide, marketed as ESIDRIX®, methylchtothiazide, marketed as ENDURON®, and benzthiazide, marketed as EXNA®;

nitrates, such as isosorbide-5-mononitrate, marketed as IMDUR®;

thromboxane antagonists, such as seratrodast, picotamide and ramatroban;

platelet aggregation inhibitors, such as cilostazol, abciximab, limaprost, eptifibatide, and CT-50547;

cyclooxygenase inhibitors, such as meloxicam, rofecoxib and celecoxib;

B-type natriuretic peptides, such as nesiritide and ularitide;

NV1FGF modulators, such as XRP0038;

HT1B/5-HT2A antagonists, such as SL65.0472;

guanylate cyclase activators, such as ataciguat (HMR1766) and HMR1069;
e-NOS transcription enhancers, such as AVE9488 and AVE3085;

anti-atherogenics, such as AGI-1067, which is in development for atherosclerosis:

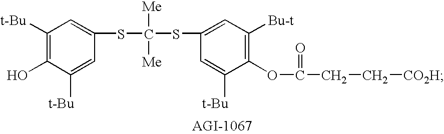

CPU inhibiters, such as AZD9684, which is in development for thrombosis;

renin inhibitors such as aliskirin, marketed as RASILEZ, and VNP489;

inhibitors of adenosine diphosphate (“ADP”) induced platelet aggregation, such as clopidogrel, marketed as PLAVIX®, ticlopidine, marketed as TICLID®, prasugrel, and AZD6140, which is in development for arterial thrombosis:

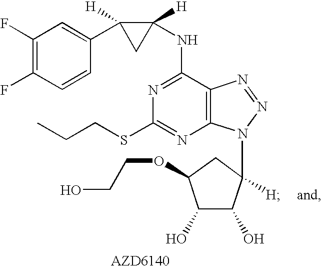

NHE-1 inhibitors, such as AVE4454 and AVE4890.

All of the pharmaceutically acceptable free base and salt forms of the above-listed cardiovascular agents are within the scope of the present invention.

In the combinations of the present invention of at least one thrombin receptor antagonist and one or more other therapeutically effective agents, the two or more active components may each be formulated individually and co-administered simultaneously or sequentially. The components of the combination can be administered individually or together in any conventional dosage form such as capsule, tablet, powder, cachet, suspension, solution, suppository, nasal spray, etc.

Alternatively, the active agents may be formulated in a single fixed-dose pharmaceutical composition comprising a thrombin receptor antagonist and the other therapeutically effective agent(s) along with a pharmaceutically acceptable carrier.

In this specification, the term “at least one thrombin receptor antagonist” means that one more different compounds that are active as thrombin receptor antagonists may be used in a pharmaceutical combination or method of treatment. Preferably, one to three thrombin receptor antagonists are used. Similarly, the term “one or more additional cardiovascular agents” means that one to three additional drugs may be administered in combination with a thrombin receptor antagonist; preferably, one cardiovascular agent is administered in combination with one thrombin receptor antagonist.

Formulations and Dosing

For preparing pharmaceutical compositions from the compounds described by this invention, an inert, pharmaceutically acceptable carrier can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), The Science and Practice of Pharmacy, 20^{th} Edition, Lippincott Williams & Wilkins, Baltimore, Md., (2000).

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g., nitrogen.

Also included are solid form preparations which are intended to be converted, shortly before use, to liquid form preparations for either oral or parenteral administration. Such liquid forms include solutions, suspensions and emulsions.

The combinations of the invention may also be deliverable transdermally. The transdermal compositions can take the form of creams, lotions, aerosols and/or emulsions and can be included in a transdermal patch of the matrix or reservoir type as are conventional in the art for this purpose.

The administration of the above-described combinations is carried out by specifying not only the formulations, but also the modes of administration and the dosing regimen.

Preferably, the formulations are solid and designed for oral administration. Orally dissolving formulations of thrombin receptor antagonists are disclosed in U.S. provisional application No. 60/689,207, which is herein incorporated in its entirety by reference.

Preferably, the pharmaceutical preparation is in a unit dosage form. In such form, the preparation is subdivided into suitably sized unit doses containing appropriate quantities of the active component, e.g., an effective amount to achieve the desired purpose.

The dosing regimen for the above-described thrombin receptor antagonists may comprise a loading dose followed by a series of maintenance doses. As a monotherapy, loading doses of 20 and 40 mgs. and maintenance doses of 1 and 2.5 mg. of compound A are preferred. More preferably, monotherapy includes doses of 40 mg. and 2.5 mg. for the loading and maintenance doses, respectively. In combination therapy, it is possible that lower doses may be favored.

Thus, in combination therapy, the loading dose of the thrombin receptor antagonist is about 5 to about 50 mg., preferably about 10 to about 40 mg. The daily maintenance dose of the thrombin receptor antagonist is about 0.5 to about 10 mg., preferably about 1 to about 5 mg. The dosage of the other therapeutically active agent(s) may range from 1 to 1000 mg per dose.

The amount and frequency of administration of the compounds of the invention and/or the pharmaceutically acceptable salts thereof will be regulated according to the judgment of the attending clinician considering such factors as age, condition and size of the patient as well as severity of the symptoms being treated.

Conditions to be Treated

The present invention also encompasses methods of treating conditions by administration of the above-described combinations. Among the conditions that may be treated (or prevented) with these combinations are cardiovascular or circulatory diseases or conditions, such as acute coronary syndrome, secondary prevention, peripheral arterial disease, thrombosis, atherosclerosis, restenosis, hypertension, angina pectoris, arrhythmia, heart failure, myocardial infarction, glomerulonephritis, thrombotic stroke, thromboembolic stroke, deep vein thrombosis, venous thromboembolism, a cardiovascular disease associated with hormone replacement therapy, disseminated intravascular coagulation syndrome, renal ischemia, cerebral stroke, cerebral ischemia, cerebral infarction, migraine, renal vascular homeostasis or erectile dysfunction.

In this context, the treatment of acute coronary syndrome is the reduction of thrombotic vascular events in at-risk patients identified by acute coronary syndrome, including unstable angina/non-ST segment elevation myocardial infarction (MI). It includes acute protection followed by maintenance.

Secondary prevention is the reduction of thrombotic vascular events in at-risk patients identified by one or more of such factors as: acute coronary syndrome (including unstable angina/non-ST segment elevation and/or MI); a history of coronary artery disease (prior MI, CABG, PCI); a history of stroke; and established peripheral arterial disease.

Peripheral arterial disease (“PAD”) is a chronic condition that results from narrowing of the vessels that supply oxygen-rich blood to the legs, abdomen, pelvis, arms, or neck. Peripheral arterial disease is also called peripheral vascular disease.

While the present invention has been described in conjunction with the specific embodiments set forth above, many alternatives, modifications and variations thereof will be apparent to those of ordinary skill in the art. Thus, for example, classes of known cardiovascular agents not recited above are within the scope of the invention, as are known but unrecited species of recited classes of cardiovascular agents. Similarly, the treatment of known but unrecited cardiovascular conditions is within the scope of the invention. All such alternatives, modifications, and variations are intended to fall within the spirit and scope of the present invention.

We claim:

1. A pharmaceutical composition comprising:
an effective amount of at least one thrombin receptor antagonist;
an effective amount of at least one cardiovascular agent selected from the group consisting of calcium channel blockers, statins, cholesterol absorption inhibitors, low molecular weight heparins, antiarrhythmic agents, alpha adrenergic agonists, beta adrenergic blocking agents, aldosterone antagonists, angiotensin-converting-enzyme (“ACE”) inhibitors, ACE/NEP inhibitors, angiotensin II receptor blockers (“ARBs”), endothelin antagonists, neutral endopeptidase inhibitors, phosphodiesterase inhibitors, fibrinolytics, GP IIb/IIIa antagonists, direct thrombin inhibitors, indirect thrombin inhibitors, lipoprotein associated phospholipase A2 (“LLpPLA₂”) modulators, direct factor Xₐ inhibitors, indirect factor Xₐ inhibitors, indirect factor Xₐ/IIₐ inhibitors, diuretics, nitrates, thromboxane antagonists, platelet aggregations inhibitors, cyclooxygenase inhibitors, B-type natriuretic peptides, NV1FGF modulators, HT1B/5-HT2A antagonists, guanylate cyclase activators, e-NOS transcription enhancers, anti-atherogenics, CPU inhibiters, renin inhibitors, inhibitors of adenosine diphosphate (“ADP”)-induced platelet aggregation, and NHE-1 inhibitors; and,
a pharmaceutically acceptable carrier for the treatment of a condition in a mammal.

2. The pharmaceutical composition of claim 1 wherein said thrombin receptor antagonist is selected from the group consisting of 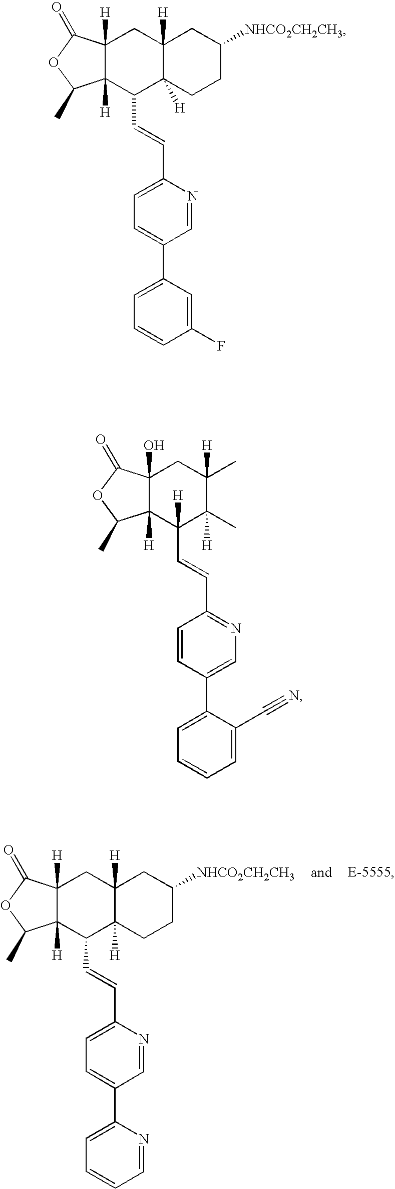
or a pharmaceutically acceptable isomer, salt, solvate or co-crystal thereof.

3. The pharmaceutical composition of claim 1 wherein said thrombin receptor antagonist is 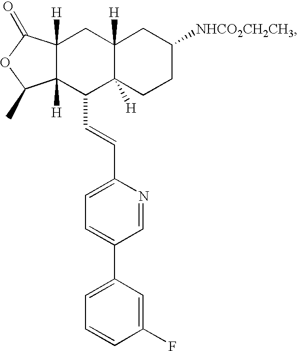
or a pharmaceutically acceptable isomer, salt, solvate or co-crystal thereof.

4. The pharmaceutical composition of claim 3 wherein said thrombin receptor antagonist is the bisulfate salt of

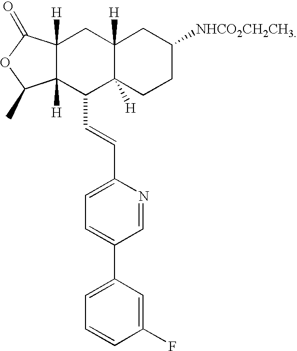

5. The pharmaceutical composition of claim 3 wherein said cardiovascular agent is a calcium channel blocker selected from the group consisting of amiodipine, felodipine, diltiazem, verapamil, nifedipine, nicardipine, nisoldipine, bepridil, and verapamil.

6. The pharmaceutical composition of claim 3 wherein said cardiovascular agent is a statin selected from the group consisting of atorvastatin, fluvastatin, lovastatin, pitavastatin, pravastatin, rosuvastatin, and simvastatin.

7. The pharmaceutical composition of claim 3 wherein said cardiovascular agent is a low molecular weight heparin selected from the group consisting of dalteparin, ardeparin, certoparin, enoxaparin, parnaparin, tinzaparin, reviparin, nadroparin, warfarin, ximelagatran, fondaparin, and enoxaparin.

8. The pharmaceutical composition of claim 3 wherein said cardiovascular agent is an antiarrhythmic agent selected from the group consisting of dofetilide, ibutilide, metoprolol, propranolol, atenolol, ajmaline, disopyramide, prajmaline, procainamide, quinidine, sparteine; aprindine, lidocaine, mexiletine, tocamide, encamide, flecamide, lorcamide, moricizine, propafenone, acebutolol, pindolol, amiodarone, bretylium, bunaftine, dofetilide, sotalol, adenosine, atropine and digoxin.

9. The pharmaceutical composition of claim 3 wherein said cardiovascular agent is an alpha adrenergic agonist selected from the group consisting of doxazosin, terazoson and prazosin.

10. The pharmaceutical composition of claim 3 wherein said cardiovascular agent is a beta adrenergic blocking agent selected from the group consisting of carvedilol, propranolol, timolol, nadolol, atenolol, metoprolol, bisoprolol, nebivolol, betaxolol, acebutolol, and bisoprolol.

11. The pharmaceutical composition of claim 3 wherein said cardiovascular agent is an ACE inhibitor selected from the group consisting of moexipril, quinapril ramipril, lisinopril, benazapril, enalapril, captopril, spirapril, perindopril, fosinopril and trandolapril.

12. The pharmaceutical composition of claim 3 wherein said cardiovascular agent is an ARB selected from the group consisting of olmesartan, candesartan, valsartan, telmisartan, irbesartan, losartan and eprosartan.

13. The pharmaceutical composition of claim 3 wherein said cardiovascular agent is an endothelin antagonist selected from the group consisting of tezosentan, bosentan, and sitaxsentan.

14. The pharmaceutical composition of claim 3 wherein said cardiovascular agent is a direct thrombin inhibitor selected from the group consisting of ximelagatran and AZD0837.

15. The pharmaceutical composition of claim 3 wherein said cardiovascular agent is a direct factor Xₐ inhibitor selected from the group consisting of fondaparinux, apixaban, razaxaban, rivaroxaban, KFA-1982, DX-9065a, AVE3247, otamixaban, AVE6324 and SAR377142.

16. The pharmaceutical composition of claim 3 wherein said cardiovascular agent is an inhibitor of adenosine diphosphate (“ADP”)-induced platelet aggregation selected from the group consisting of clopidogrel, ticlopidine, prasugrel, and AZD6140.

17. The pharmaceutical composition of claim 3 wherein said cardiovascular agent is a cholesterol absorption inhibitor selected from the group consisting of ezetimibe and AZD4121.

18. A method of treating or preventing a cardiovascular condition in a mammal in need of said treating comprising administering to said mammal a pharmaceutical composition of any of claims 1-17, wherein said cardiovascular condition is acute coronary syndrome, secondary prevention, peripheral arterial disease, thrombosis, atherosclerosis, restenosis, hypertension, angina pectoris, arrhythmia, heart failure, myocardial infarction, glomerulonephritis, thrombotic stroke, thromboembolic stroke, deep vein thrombosis, venous thromboembolism, a cardiovascular disease associated with hormone replacement therapy, disseminated intravascular coagulation syndrome, renal ischemia, cerebral stroke, cerebral ischemia, cerebral infarction, migraine, renal vascular homeostasis or erectile dysfunction.

19. A method of treating or preventing a cardiovascular condition in a mammal in need of said treating comprising administering to said mammal a first pharmaceutical composition comprising a thrombin receptor antagonist and a second pharmaceutical composition comprising a cardiovascular agent.

20. The method of claim 19 wherein said thrombin receptor antagonist is 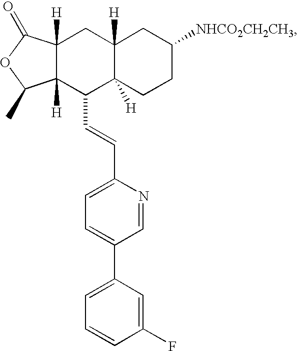
or a pharmaceutically acceptable isomer, salt, solvate or co-crystal thereof.

21. The method of claim 20 wherein said cardiovascular agent is selected from the group consisting of calcium channel blockers, statins, cholesterol absorption inhibitors, low molecular weight heparins, antiarrhythmic agents, alpha adrenergic agonists, beta adrenergic blocking agents, aldosterone antagonists, angiotensin-converting-enzyme (“ACE”) inhibitors, ACEINEP inhibitors, angiotensin II receptor blockers (“ARBs”), endothelin antagonists, neutral endopeptidase inhibitors, phosphodiesterase inhibitors, fibrinolytics, GP IIb/IIIa antagonists, direct thrombin inhibitors, indirect thrombin inhibitors, lipoprotein-associated phospholipase A2 (“LpPLA₂”) modulators, direct factor Xₐ inhibitors, indirect factor Xₐ inhibitors, indirect factor Xₐ/IIₐ inhibitors, diuretics, nitrates, thromboxane antagonists, platelet aggregations inhibitors, cyclooxygenase inhibitors, B-type natriuretic peptides, NV1FGF modulators, HT1B/5-HT2A antagonists, guanylate cyclase activators, e-NOS transcription enhancers, anti-atherogenics, CPU inhibiters, renin inhibitors, inhibitors of adenosine diphosphate (“ADP”)-induced platelet aggregation, and NHE-1 inhibitors.
